(19) United States
(12) Patent Application Publication
(10) Pub. No.: US 2012/0156779 A1
(43) Pub. Date: Jun. 21, 2012

(54) METHOD FOR CELL EXPANSION

(75) Inventors: Cecilia Anneren, Uppsale (SE); Andreas Axen, Uppsala (SE); Asa Bjurling, Uppsala (SE); Christine Sund Lundstrom, Uppsala (SE); James Van Alstine, Uppsala (SE); Gunnar Froman, Uppsala (SE)

(73) Assignee: GE HEALTHCARE BIO-SCIENCES AB, UPPSALA (SE)

(21) Appl. No.: 13/392,538

(22) PCT Filed: Aug. 23, 2010
(86) PCT No.: PCT/SE2010/050905

§ 371 Date: Feb. 27, 2012

(30) Foreign Application Priority Data
Aug. 27, 2009 (SE) 0950617-1

(51) Int. Cl. C12N 5/02

(52) U.S. Cl. 435/378

(57) ABSTRACT

The present invention relates to a method for cell expansion. In the method, preferably a cell culture product is used, such as a microcarrier, or other adherent cell culture surface, comprising degradable polysaccharide, preferably starch, modified with small molecular weight cell-binding ligands. This allows recovery (detachment) of adhered cells to be aided by degradation of the culture surface with enzymatic agents, such as amylase. The method for cell expansion comprises the following steps: a) adding cells, culture medium and cell culture surface comprising a degradable polysaccharide with guanidine group containing ligands to a bioreactor; b) expanding said cells by adherent cell culture; and c) aiding the detachment of said cells by exposing them to a polysaccharidase to degrade the culturing surface.

### CROSS-REFERENCE TO RELATED APPLICATIONS

This application is a filing under 35 U.S.C. §371 and claims priority to international patent application number PCT/SE2010/050905 filed Aug. 23, 2010, published on Mar. 3, 2011, as WO 2011/025445, which claims priority to patent application number 0950617-1 filed in Sweden on Aug. 27, 2009.

### FIELD OF THE INVENTION

The present invention relates to a method for cell expansion. In the method, preferably a cell culture product is used, such as a microcarrier, or other adherent cell culture surface, comprising degradable polysaccharide modified with small molecular weight cell-binding ligands. This allows recovery (detachment) of adhered cells to be aided by degradation of the culture surface with enzymatic agents, which do not have protein substrates and therefore cause less alteration of the cultured cells.

### BACKGROUND OF THE INVENTION

Cell culture techniques are vital to the study of animal cell structure, function and differentiation as well as for the production of many important biological materials, such as enzymes, hormones, antibodies, nucleic acids, virus vaccines, and viral vectors for gene therapy. Another important area for cell culture is cell expansion, from a small to a large cell population, as used for example in cell therapy. Ideally the cultured cells should not be altered by culture or related cell recovery processes.

It is often necessary to culture cells on a cell adhering surface since growth of many mammalian and certain other cells is anchorage-dependent. Conventional adherent cell culture on the surfaces of tissue culture bottles, vials, well slides or other vessels gives a limited yield of cells due to the small surface area to volume ratios of such vessels.

Microcarrier culture involves growing adherent cells as mono layers on the surface of small, micron range diameter particles which are usually suspended in culture medium by gentle stirring. Microcarrier suspension culture systems are readily scalable and make it possible to achieve yields of several million cells per millilitre. Microcarrier culture has made it more economically feasible to use adherent cells for production of vaccines and some other biotechnical products. Cells can be grown on microcarriers in a variety of formats such as suspended in spinner flasks, packed in column beds (perfusion culture) or even on microcarriers in micro titre plate wells.

Commercial microcarriers are often produced using cross linked polymers of dextran, cellulose, polyethylene or other polymers. Some such products feature polymeric coatings on glass or other net negative charged surfaces. Most cells exhibit significant net negative charge due to abundant surface carboxylic acid groups. This makes it easier for them to attach and grow at net positive charged surfaces. In many cases growth surfaces are modified with positively charged entities to promote carrier surface adherence of cells. Examples include commercially available Cytodex 1 microcarriers, prepared from cross linked dextran particles coated with diethylaminoethyl (DEAE) groups, as well as DEAE modified cotton (Cytopore 1) bead carriers from GE Healthcare BioSciences AB. In addition to non-specific (e.g. electrostatic) interactions, affinity or other interactions may hold cells to surfaces modified with appropriate affinity substances. Cytodex 3 microcarriers are prepared from cross linked dextran beads coated with a collagen protein layer designed to mimic the protein coated surfaces which cells bind to in the body (Microculture Cell Carrier Principles and Methods, GE

Healthcare, Application Booklet 18 1140 62, available from GE Healthcare). Cells also bind to a variety of other proteins via specific affinity interactions. Polypeptides containing the specific tripeptide RGD found in many cell binding proteins have been used as specific affinity ligand for binding cells. (U.S. Pat. No. 5,830,507A, U.S. Pat. No. 5,563,215A). Other polypeptide affinity ligands are known (e.g. WO 0153324 A1 Novel Haptotactic Peptides). DEAE is not native to biological systems and may be cytotoxic under some conditions (www Toxnet ref for DEAE CASRN 100-37-8). One possible advantage of natural protein or synthetic polymers of amino acids over small molecular weight ligands such as DEAE, relates to the biocompatible nature of such natural and synthetic proteins (e.g. see Biomaterials volume 24 (2003) pages 4253-4264, The design of polymer microcarrier surfaces for enhanced cell growth Dai Katoa, Masahiko Takeuchia, Toshihiko Sakuraia, Shin-ichi Furukawab, Hiroshi Mizokamib, Masayo Sakataa, Chuichi Hirayamaa, Masashi Kunitakea). However there are still concerns related to (virus or prion containing) animal sources of such proteins, as well as leakage of such carrier associated proteins into bioprocess feed streams.

Recently filed patent application SE 0802474-7 related to cell culture surfaces modified with a series of positively charged ligands which mimic the charge based cell binding performance of DEAE or similar ligands but are formed using arginine or related cationic compounds containing guanidine groups. This SE-application provides a general method of attaching Arg type ligands to microcarrier surfaces, which appears to yield effective cell carriers when used with a wide a variety of guanidine group containing or similar ligands. This is important for, as noted in SE 0802474-7, it is not always possible to predict if such ligands or their method of surface coupling will result in surfaces which support cell growth. Thus U.S. Pat. No. 6,929,818 B2 (Methods and clinical devices for the inhibition or prevention of mammalian cell growth) describes inhibition of mammalian cell growth at biomedical surfaces associated with at least one biguanide group.

In order to recover adherent cells from culture surfaces their cell to surface interactions must be weakened. Typical approaches include Mechanical (shearing), Chemical e.g treatment of cells with ethylene diamine tetra acetic acid (EDTA) or other chelator of the divalent cations which help to stabilize cell membrane structure, Osmotic (hypo-osmotic solutions to promote cell geometry changes), or Enzymatic. The latter typically involves the use of nonspecific protein hydrolases such as trypsin, chymotrypsin, papain, etc. The most effective and common approach involves trypsinization. Such treatment leads to a widespread and nonspecific alteration of cell-carrier interface including cell associated protein surfaces. Two drawbacks of enzymatic treatment are introduction of foreign protein into culture solutions (which has led to commercialization of non-animal derived, papain or recombinant trypsin products) and negative effects on cell surfaces. The latter can include phenotypic changes or even cell death.

It would be better if cells could be released from cell carrier surfaces using either reduced or no specific protease treatment. This especially true for stem and other medical cell based therapies where such treatment may be related to phenotype changes. Although cross-linked dextran (e.g. Sephadex™ or Cytodex™) microparticles are known to be fairly biocompatible and will dissolve slowly over time in the body their in vitro degradation can be enhanced by exposure to dextranase enzymes (U.S. Pat. No. 6,378,527B1). So too dextran coated magnetic particles for cell separation can be exposed to dextranases in order to release cells adsorbed to the particles via affinity ligands grafted to the dextran layer (WO1996031776 A1). Attempts have also been made to oxidise DEAE modified cross linked dextran (DEAE Sephadex) particles to make them more readily hydrolysed in vivo (C. Christoforu, et al., J. Biomaterials Res. 376-385, 1998). For some applications, it may be possible to incorporate enzymes directly into cell carriers or separation beads so as to promote breakdown of such solid supports (e.g. U.S. Pat. No. 5,160,745A).

U.S. Pat. No. 6,184,011B1 notes use of various polysaccharidase enzymes to degrade polysaccharide based particles to aid “cell testing and separation methods to meet the needs of the food, medical, environmental and veterinary industries”. Similar approaches may be suitable for cell culture and analysis related to applications such as food pathogen analysis, but are expected to be limited (due to concerns related to foreign proteins or cell alteration) in regard to biopharmaceutical production or cell therapy.

Molday et al (U.S. Pat. No. 4,452,773A) relates to use of dextran polysaccharide based surface coating on magnetic beads for cell separation via specific surface affinity interactions such as antibody mediated immuno-affinity separation. Molday et al used oxidation to promote transformation of dextran hydroxyls to dialdehyde groups so as to enhance reactive groups for affinity ligand grafting.

U.S. Pat. No. 5,563,215 describes a substrate for growing cells comprising a base material, preferably polymer chosen from polystyrene, polypropylene, polyethylene terephthalate, polyallomer, cellulose acetate, and polymethylpentene., with an (oxidized) dialdehyde starch (DAS) coating to which is attached a cell binding oligopeptide selected from the group consisting of Gly-Arg-Gly-Asp-Ser-Pro-Lys, Lys (SEQ ID NO. 1), Lys-Gly, Gly-Gly-Tyr-Arg (SEQ ID NO. 2), and Arg-Lys-Asp-Val-Tyr (SEQ ID NO. 3). The oligopeptides were typically bound to the aldehydic DAS groups via either the peptide alpha-amine or epsilon-amine of lysyl residues. Such an approach has various drawbacks. First the oxidation can be difficult to control and only generates aldehyde groups, which are not very reactive to the target amine groups. Secondly, as noted in the patent, the oligopeptide grafting reaction often requires a reducing agent reaction (the third step including starch activation by oxidation) such as NaBH4, or NaCNBH3 to reduce the (Schiff's base) imine produced in the first reaction to a more stable carbon to nitrogen bond. Such reducing agents are expensive, require special handling (e.g. to reduce operator exposure to such agents, and exposure of such reagents to moisture), and are highly reactive and therefore difficult (but not impossible) to handle at larger scale. A more commercially suitable process would involve more controllable, water based reaction, of stable and relatively less expensive reagents easier to handle at large scale.

For many therapeutic and other high value applications it would be good to have carrier surfaces degraded by proteins which like have less effect on cell surfaces than proteases, and like amylases occur naturally in human tissues. It would also t be good to have carrier surfaces based on naturally occurring biocompatible polysaccharides.

### SUMMARY OF THE INVENTION

The present invention provides a method for cell expansion using novel microcarriers for cell culture for expanding cell types such as MDCK cells and Vero cells for use in protein and virus expansion applications as well as for providing expanded cultures of stem cells and other cells for therapy.

The invention provides degradable microcarriers preferably based on starch hydrogel particles, or starch coatings, provided with arginine (Arg) or analogous ligands to promote cell attachment and allow for normal cell growth in culture. It was found that it is possible to modify the starch hydrogel with these ligands via bifunctional reagent in manner such that

a. There is no need for costly and potentially dangerous chemical reduction methods such as via use of use of NaBH4, or NaCNBH3.

b. The ligands strongly promote cell attachment and proliferation on the starch surfaces.

c. The related starch gel activation and ligand grafting chemistry can be controlled so that it does not eliminate the ability of cells to be cultured.

d. The related starch gel activation and ligand grafting chemistry can be controlled so that surfaces offering good culture performance are also be amenable to amylase enzyme mediated degradation.

e. The gel activation can be controlled in manner to influence susceptibility of the gel to amylase catalysed degradation.

The inventors have found that starch would be particularly beneficial for situations where one wishes to deliver an expanded set of cultured cells into the body on a carrier which breaks down in the body. For different applications it would be good to have control over the rate the cell bearing particles or surfaces are biodegraded. Given the occurrence of amylase in the body starch particles might be suitable candidates. However in the case of starch particles the major problem is that cells do not typically bind to their surfaces.

The invention relates to a method for cell expansion comprising the following steps: a) adding cells, culture medium and cell culture surface comprising a degradable polysaccharide, having arginine (Arg) or other guanidine group containing ligands on its outer surface, to a bioreactor; b) expanding said cells by adherent cell culture; and c) aiding the detachment of said cells by exposing them to a polysaccharidase or other agent which enzymatically directed to degrade the culturing surface.

The culture surface is preferably a microcarrier but may also be a slide, a biosensor chip, a disposable tube or bag, a microtiter plate, or other object whose surface is capable of supporting the adherent cell culture layer.

In a preferred embodiment, the degradable polysaccharide is coated to the microcarriers or other culture surfaces.

The coating and the microcarrier may be made of different material, such as different polysaccharides, for example Cytodex (i. e. dextran) with a starch-coating. The materials may be chemically cross-linked to provide stability, porosity, density or other functional properties.

According to the invention, the microcarrier comprises the degradable polysaccharide and only the surface thereof has been provided with ligands.

The polysaccharide may for example be dextran or starch and the polysaccharidase is dextranase or amylase.

The guanidine group-containing ligands are Arginine-ligands, preferably monopeptides or dipeptides comprising at least one arginine residue.

The ligands are preferably covalently grafted to the culture surface which has been activated with a bifunctional reagent (which allows the correct practical functioning of the other components for adherent cell culture). The inner part of the microcarrier does not contain any ligands. This enhances amylase degradation.

Preferably the ligands are attached to the degrading polysaccharide surface via an allylglycidylether or analogous bifunctional reagent which is first coupled to the carrier surface, or to the ligand.

The cultured cells may also be detached by a method involving polysaccharidase which is not added to the cultured cells environment but occurs spontaneously as a recombinant or normal cell gene product.

The microcarriers may be provided with magnetic particles to facilitate separation of the cells. Also other entities may be included providing additional separation, diagnostic, reporter, or imaging capabilities.

The in vitro cell removal by amylase or other polysaccharidase may be enhanced by use of various enzymatic dissociation agents such as trypsins, collagenases or combination products e.g. Accumax, which combines protease, collagenolytic and DNase activities.

The microcarriers may be made solely of polysaccharide and ligands. Alternatively, the degradable polysaccharide may be coated to the microcarriers in which case the microcarrier may have a core of any other suitable material, such as cotton or a synthetic polymer or other chemicals or sub particles embedded in a matrix. The latter case gives an opportunity to increase the stability or functionality of the microcarriers, e.g. with magnetic or other properties.

Preferably, the Arg-ligands are simply coupled arginine but they can be dipeptides comprising at least one arginine residue. They can also be other groups containing guanidine functionalities.

The cells cultivated in the method of the invention may be primary cells or stem cells. But also established cell lines, for example, Vero or so called MDCK cells for virus production.

The method may comprise a step of decanting of culture medium before step c) and in this case it is preferred that the microcarriers are provided with magnetic particles. Thus, in any cell cultivation situation where sedimentation is desired, the sedimentation of the microcarriers may be enhanced by adding sub-particles which are denser or in cases where magnetic properties shall aid carrier handling the sub-particles can be magnetic particles, such as Fe₂O₃. Various such secondary properties can be combined thus magnetic sub particles embedded in the carrier particle might serve to enhance particle isolation, before or after cell removal, as well as offer various medical imaging capabilities. This raises the possibility of the carriers being used to both expand stem or other cells in vitro and then deliver them in vivo.

The ligand modified starch hydrogels do not just have to be used as carrier particles or coatings for carrier particles. They can be used as coatings for variety of other surfaces which cells may be cultured on in regard to miscellaneous expansion, sensing, diagnostic or other applications. These include micro-titre plate or well slide surfaces, biosensor surfaces, biochip surfaces, optical surfaces, etc.

Another interesting aspect of the invention is that for various applications the starch hydrogel particles or coatings may be desired to offer various combinations of abilities to bind and culture cells (i.e. provide a biocompatible surface for normal cell behaviour) as well as able to be degraded by enzymes which are directed to hydrolyse the starch hydrogel to various degrees. The invention provides a way to exert some control over these properties based on the use of different relative amounts of coupling reagent and ligand, as well as if the particle is composed entirely of starch or simply a starch coating, and if the coating is modified chemically throughout, or only modified at the external surface in a so called “lid” synthesis such as is described in U.S. Pat. No. 6,572,766.

### BRIEF DESCRIPTION OF THE DRAWINGS

FIG. 1 schematically shows chemical coupling of guanidine containing ligand to polyscaccharide bead or other gel surface containing hydroxyl groups via a bifunctional reagent. In this example the reactive surface is a bead, the ligand is arginine amino acid, and the bifunctional reagent is allylglycidylether.

FIG. 2 shows the coupling of 2-diethylamino ethyl chloride hydrochloride to a hydroxyl group possessing matrix under basic conditions. Included is also the di-coupling of 2-diethylamino ethyl chloride hydrochloride to the tertiary amine of an already couple DEAE group. This is a side reaction that always takes place to a larger or smaller extent with the used coupling conditions.

FIG. 3 shows a graph of carrier bead density and swelling versus degradation time for starch beads indicating that of amount of dry material in beads and degree of cross linking influence the degradability.

FIG. 4 shows a graph of carrier allylation levels versus arginine ligand coupling levels. The maximum level of arginine that can be coupled is in each case directly dependent on the corresponding allylation level.

FIG. 5 shows a graph of the effect of chemical modification on starch carrier performance in regard to culture of human mesenchymal stem cells and amylase degradation of carrier. Cell attachment and growth was scored 0-5 where 5 is best and degradation was scored 0-8 where 0 is no degradability and 8 highly degradable.

FIG. 6 shows the effects on mesenchymal stem cell growth in response to different levels of amylase; and

FIG. 7 shows cell culture and degradation of starch beads coupled according to the lid-approach.

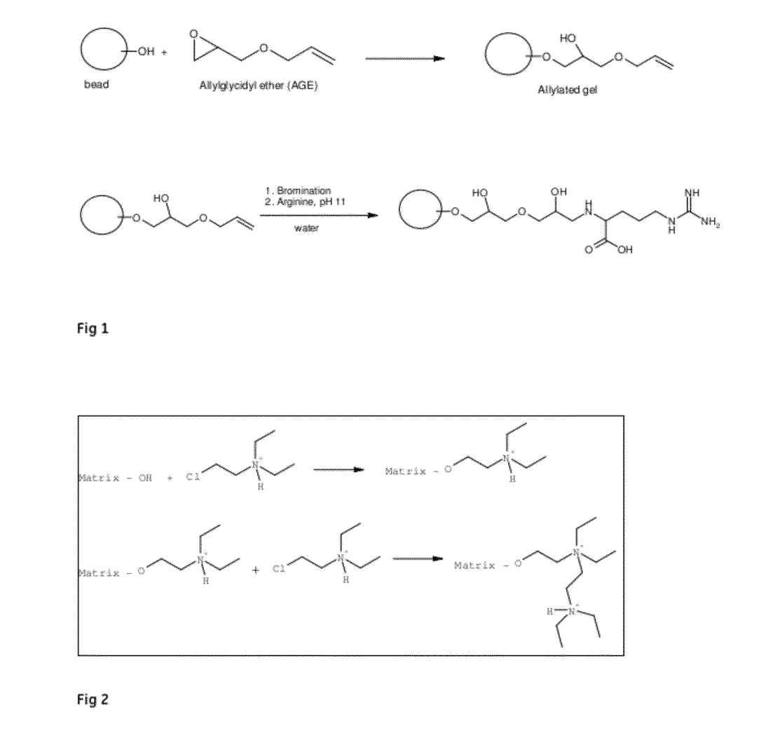

### DETAILED DESCRIPTION OF THE INVENTION

The invention will now be described more closely in association with the drawings and some non-limiting examples.

The present inventors realized that ligands based on naturally occurring chemical structures (e.g. guanidines) or biochemical substances (e.g. arginine amino acid or arginine containing peptides) may not necessarily be effective promoters of cell attachment and culture at biodegrading surfaces because the ligand attachment chemistry and resulting alteration of the hydrogel may lead to surfaces which either do not degrade or degrade too readily to be of use. They also recognized that different applications may require degrading surfaces which offer varied degrees of degradation and cell attachment, and maintenance of normal cell behaviour as exemplified by the ability to culture the cells. Examples of such different applications include carrier surfaces for culture of cell in production of vaccines (where cells may be lysed post recovery) as opposed to expansion of therapeutic cells for later delivery into a patient, or attachment and culture of cells at a biosensor or other analytical method related surface. They also recognized the importance of starch or similar hydrogel carriers or surfaces which can be degraded by amylase or similar polysaccharidases which either occur naturally (e.g. in vivo) or can be added to a culture, and which primarily act to degrade the carrier substrate not cell surface or cell matrix associated protein structures in the manner of the overt trypsinisation often used when removing cells from culture surfaces.

Some of the above concepts are summarised in Tables 1 and 2. Table 1 indicates a list of desired traits of cell carriers and related cell culture or cell localizing analytical surfaces in relation to variety of applications. The traits are then matched against both commercial cell carriers such as Cytodex 1 and Cytodex 3, as well as against various starch and starch coated cell carriers which display arginine or analogous biocompatible ligands to promote cell attachment. More details regarding such carriers are noted in Table 2, including the possibility to only display ligands at the surface of the carrier hydrogels so as to both reduce production costs and to introduce an operator controlled variable which might allow better tailoring of bead degradation, density and other properties to various applications. It is expected that cells adhered to a hydrogel surface do not normally suffer any influence from ligands or other substances embedded in the hydrogel beyond the cell to gel contact surface.

TABLE 1
Commercial and Experimental Cell Carriers Matched Against Desirable Properties

| | | | | | | Cytodex- |
|---|---|---|---|---|---|---|
| | | Cytodex 1 | Cytodex 3 | Cytodex | Starch | Starch- |
| | Property of Interest | (DEAE) | (Gelatin) | Arg | Arg | Arg |
| I | Production and Business | | | | | |
| 1 | Readily made in large scale | 3 | 3 | 3 | 1 | 3 |
| 2 | GMP Producible | 3 | 1 | 3 | 3 | 3 |
| 3 | Simple production re EHS | 3 | 3 | 3 | 3 | 3 |
| 4 | Low cost, available reagents | 3 | 1 | 3 | 1 | 3 |
| 5 | No serious QC issues | 3 | 1 | 3 | 3 | 3 |
| II | Culture Performance | | | | | |
| 1 | Suitable for Vero Cells | 3 | 3 | 3 | 3 | 3 |
| 2 | Suitable for some MSC types | 3 | 3 | 3 | 3 | 3 |
| 3 | Suitable for other SC types⁺ | 1 | 1 | 1 | 1 | 1 |
| 4 | Suitable for serum free media | 1 | 1 | ? | ? | ? |
| 5 | Keeps ES geno- + phenotype. | ? | ? | ? | ? | ? |
| 6 | No leeching toxic or bioactives. | 1 | 1 | 3 | 3 | 3 |
| 7 | Demo culture 1 to 100 L scale | 3 | 3 | ? | ? | ? |
| 8 | OK for high density culture | 3 | 3 | 3 | 3 | 3 |
| 9 | OK for broad range of cells | 3 | 3 | 3 | 3 | 3 |
| 10 | Bead density like Cyt. 1 or 3 | 3 | 3 | 3 | ? | 3 |
| III | Cell Detachment + Reculture | | | | | |
| 1 | OK for normal treatments | 3 | 3 | 3 | 3 | 3 |
| 2 | OK for enzyme-free methods | 1 | 1 | 1 | 1 | 1 |
| 3 | OK for milder or more | 0 | 0 | ? | 1 | 1 |
| | Economical conditions. | | | | | |
| 4 | Cells detach with treatment that | 1 | 1 | 1 | 3 | 3 |
| | does not kill some cells | | | | | |
| 5 | Above do not affect cell type or | ? | ? | ? | ? | ? |
| | function | | | | | |
| IV | Other Properties | | | | | |
| 1 | Injectable | 0 | 0 | 0 | 1 | 1 |
| 2 | Biodegradable | 0 | 0 | 0 | 3 | 1 |
| 3 | Possible FDA approvable | ? | 0 | 1 | 3 | 1 |

Notes
1. Representative cell types noted include Vero Cells for eukaryotic cells used in cell based production, mesenchymal stem cells (MSC) and other SCs for cell based therapy and sensing.
2. Cyt. = Cytodex. Cytodex 1 and 3 are commercial carriers. Cytodex 3 has collagen (gelatine) coating. Cytodex-Arg is arginine modified Cytodex (Sephadex) carrier. Starch-Arg is arginine modified starch particles. Cytodex-Starch-Arg is represents Starch-Arg coated carriers including Arg″ lid″ modified carriers.
3. Above properties not ranked in importance. Results noted refer to the best performing Cytodex Arg or Arg based ligands, or Magle AB starch gel Arg particles tested.
4. Results graded in regard to possible commercial usefulness in regard to various adherent cell and stem cell types so that 3 indicates no concern, 1 indicates small concern(s) or not yet demonstrated, 0 indicates significant concerns, while “?” means the answer is yet unknown..

TABLE 2
Possible Products for Cell Culture Related to the Present Invention

| Carrier or | Carrier | | | Production | |
|---|---|---|---|---|---|
| Surface | Type | Market | Possible Features | Cost | Product Line |
| Sephadex-Arg | Cytodex | Culture | GMP | + | Vary ligand or |
| | | | Biocompatible ligand | | ligand density |
| Sephadex- | Cytodex | Culture or | GMP | ++ | Vary starch |
| Starch-Arg | | Analysis | Biocompatible ligand | | degradation |
| | | | Cell remove w/o trypsin | | properties |
| Starch-Arg | Starch | Culture and | GMP | ++ | Vary starch |
| | | Delivery | Biocompatible ligand | | degradation |
| | | | Cell remove w/o trypsin | | properties |
| | | | In vivo cell delivery | | |

Notes
1. For comparison sake the first row includes Sephadex-Arg (e.g. arginine modified Cytodex) beads.
2. Sephadex-Starch-Arg is an example of a polysaccharide coated, ligand modified, carrier bead.
3. Starch-Arg is an example of a ligand modified readily degradable carrier bead where the ligand is attached either throughout the carrier matrix or only as a “lid” near its external surface.

Experimental

This section comprises Experimental Methods followed by Experimental Results and Discussion

I. Experimental Methods

A. Particle Preparation and Chemical Modification

1. Cytodex Preparation

Cytodex particles were obtained from GE Healthcare BioSciences AB, Uppsala, Sweden. Cytodex 1 and 3 are cross-linked dextran, i. e. essentially Sephadex G50 chromatography particles, modified with DEAE or gelatin surface coatings, respectively, to promote cell attachment and growth (Microculture Cell Carrier Principles and Methods, GE Healthcare, Application Booklet 18 1140 62). Basic Cytodex base matrix was Sephadex G50 media.

2. Starch Gel Preparation

Starch gel particles were obtained from Magle AB. Magle An particles are composed of partially hydrolyzed potato starch which is cross linked with epichlorohydrin. Through a well controlled production process spherical particles produced from plant starch can offer controlled size, density, and cross linking degree and, as a result, in vivo degradation times.

Starch bead samples were by a process with that the starch is exposed to acid at high temperature and pressure under a controlled time. The hydrolysed starch is then washed and dried and then treated with sodium hydroxide. In the particle production a chemical agent may be added to protect the starch from oxidation during handling. The starch is then formed into particles via use of a common emulsifier which is dissolved and added in toluene. A water-in-oil type emulsion is formed and mixed to achieve optical droplet size, at which point epichlorohydrin is added to form the particles. The suspension with starch particles is then washed with water and ethanol to remove free reagents and any other contaminants. The particles are then dried to a white powder. The resulting particles can be impervious to water degradation but are degraded by amylase activity. Various secondary modifications can affect their degradability and this can be used to optimise various properties and the efficacy of different products.

3. Activation and Ligand Coupling to Polysaccharide Hydrogel Surfaces.

3.1. Coupling of Arginine and Related Ligands to Allyl Activated Hydrogels

See FIG. 1 for basic reaction scheme used for coupling.

3.1.a Allylation Reaction:

Starch beads were mixed with water in a three-necked flask with stirrer. Na₂SO₄ was added to the flask and was dissolved for 1.5h at 30° C. NaOH 50% and allyl glycidyl ether (AGE) was added. The slurry was heated to 50° C. and the reaction was continued over night. The reaction was stopped by neutralizing with acetic acid 60%. The gel bead particle was washed with water, ethanol and finally with water.

3.1.b Coupling of Arq-Type Liqands to Allyl Activated Microcarriers

Reagents were ACS grade or better. Arginine (Arg) or related ligands can be coupled to allylated gel via the primary amine on the C2-carbon of the amino acid arginine. Drained allylated gel was transferred to a beaker and water (approximately the same amount water as the transferred drained gel volume) was added to the gel. During vigorous stirring bromine (pure bromine or bromine water) was added to a consistent yellow colour. After about 5 minutes of stirring sodium formate was added until the gel slurry was completely discoloured and then left stirring for about 15 minutes. The gel was left to sediment and the supernatant was removed. Overhead stirring was begun and NaCl solution and L-arginine was added to the gel slurry. The slurry was then left stirring at 50° C. over night. The reaction was stopped after about 18 hours and the gel washed with 0.9% NaCl.

Two different starch gel particle batch samples of different degradation time and density (C1 and C2 see above) were also activated with epoxide and then grafted with arginine to 0.52 mmol per g.

3.2. Lid Bead Variants of AGE Activated Arginine Ligand Modified Starch Particles.

One example is given below. Various amounts of added bromine was used in order to vary the thickness of the ligand lid.

To 9 grams of drained allylated gel was added 150 of water and 1 gram of sodium acetate trihydrate. The slurry was stirred and 50 mL of water to which 8 μL of bromine had been added was added in 6 portions. Directly thereafter the gel was washed with a 11% sodium chloride solution on a glass filter. The gel was then transferred to a 100 mL flask and 1.1 gram of arginine and the slurry was stirred for 16 hours at 50° C. Finally the gel was washed on a glass filter using 1% sodium chloride followed by water. Elemental analysis indicated a ligand density of 0.08 mmol/g gel.

3.3. Coupling of Arginine Ligands to Hydrogel Via Epichlorohydrin

2 g starch beads were swelled in 64 ml water during stirring. 8.0 ml NaOH 50% were added and the slurry was cooled to 21° C. 30 ml epichlorohydrin (ECH) was added during 2 h (0.25 ml/min). After the addition was completed the reaction was left for 2h before the gel was washed with water on a glass filter. The epoxy content was measured according to the titration method noted in SE 0802474-7. To 70 ml of the gel 9.0 ml water and 0.9 g arginine were added during stirring, the temperature was increased to 45° C. and the reaction was continued over night. The gel was washed with 8 gel volumes 0.9% NaCl.

3.4. Coupling of DEAE (2-diethylamino Ethyl Chloride Hydrochloride) Ligands

Two methods were used for coupling DEAE to starch beads

3.3.a Method 1. Toluene and bensetonchloride were mixed. Starch beads were added to the flask and the slurry was stirred for 15 min. A mixture of water, NaOH and NaBH₄ was prepared and added to the slurry together with water. Stirring continued for 2h. 2-diethylamino ethyl chloride hydrochloride was added with water and stirring continued for 1h. The temperature was increased to 60° C. and the reaction was left over night. The beads were washed with NaCl 0.9% solution.

3.3.b Method 2 Water was added to starch beads and the beads were left to swell for 5 minutes. Under stirring NaOH 50% and NaBH₄ were added. More NaOH 50% and Na₂SO₄ were added and the temperature of the slurry was fixed to 27° C. and left with stirring for 1h.

2-diethylamino ethyl chloride hydrochloride was added and the reaction was left over night. The reaction was neutralized with HCL and washed with NaCl 0.9% solution.

The cell attachment and proliferation rate were compared with starch beads modified using DEAE as ligand as well as with Cytodex 1 and 3. It was found that the DEAE ligand could not promote cell attachment/growth on starch beads while Arg allowed cells to attach and expand in an as high rate as Cytodex 1 and 3, i.e. DEAE on dextran beads.

B. Density of Starch Particles

The density of the starch beads was determined in a Percoll (GE Healthcare) gradient adjusted to physiological conditions and with Density Marker Beads (GE Healthcare) as control. The density of the base-matrices followed the degradability; the longer degradability half-time the higher density. Cytodex 1 and 3 have a density of 1.03 and 1,04 g/ml, respectively and served as controls. Ligand coupling to the base matrices only had limited effects on the density of the starch beads.

C. Amylase Mediated Degradation of Starch Particles and Coatings

C.1 The method Magle AB uses to rank the degradability half-time of starch beads involves degrading 6 mg beads (approx 80 μl swelled gel) in 20 ml 150 mM NaCl, 10 mM NaPhosphate pH 7 (PBS) and measuring free glucose after a 25 min degradation period. Such data is given in Table 3. The draw-back with this method is that one does not follow the carriers until they are fully degraded, and the carriers are diluted to degree which may not occur in in vivo based applications.

C.2 GE Healthcare method was developed to address some functional concerns in the above method. It involves the following degradation protocol; 20 μl 50:50% bead slurry was degraded in 700 μl Triton X-100 in PBS and 3.1 U amylase/ml with intermittent mixture. We followed degradation over time (up to 70 hours) by looking in the microscope and scored the degradability according to the criteria below.

Degradation Score 0 to 8

(for 3.1 U amylase/ml, in 700 μl and 20 μl bead slurry at 50% v/v)

0=no degradation at any time-point or concentrations in shape) and almost degraded at 70 h.

1=some minor changes in appearance (looking smooth) after 40 h nothing more happens.

2=some minor changes in appearance (smooth) at around 20 h, clear changes at 40 h (ghost or change in shape) and almost degraded at 70 h

3=some minor changes at 8 hours (smooth), clear changes in appearance (ghost) around 20 h, almost degraded at 40 h

4=minor changes smooth at 2 h, clear changes in appearance (ghost or change in shape) around 8h and almost degraded at around 20 h

5=clear changes in appearance (ghost or change in shape) at 2 h, degraded at 8 hours

6=some degradation even without amylase. Starts to degrade at once with amylase and almost degraded by 5 h

7=A lot of degradation without amylase: Half-time with amylase 2.5 h

8=Half-time with amylase around 1 hour or less

D. Cell Culture Methods

Before adding the cells, the microcarriers were washed twice with basal medium. 40 μl of a 50:50% bead slurry (Approximately 5000 beads) and 800 μl media were added to each well in a 24 well plate and equilibrated at 37° C., 5% CO₂ for at least 1 hour. 20 000 cells in suspension were then added to each well. Cell attachment and spreading was studied in the microscope at 4, 24, 48 and 72 hours. Notes and photos were taken and cell attachment and growth was scored as follows:

Cell Attachment and Growth Ranking

0=No attachment

2=Attachment but no spreading

3=Attachment and spreading but less growth compared to Cytodex 1 over 72 hours

4=Equal growth compared to Cytodex 1 over 72 hours

5=Better growth than Cytodex 1 over 72 hours

D.1 Vero Cells

Vero cells were cultured in Dulbecos Modified Eagles Medium (DMEM), 10% Foetal Calf Serum (FCS) and 10 mM Hepes buffer from Sigma Aldrich or similar vendor.

D.2 Human Mesenchymal Cells

Human mesenchymal stem cells were purchased from Lonza (cat PT-2501) and cultured in the recommended mesenchymal cell growth medium, MSCGM (PT-3238 and PT-4106E) according to the manufacturer's instruction to 80% confluency. Recommended seeding density was approximately 5000 cells/cm². The cells had to be sub cultivated once a week for three times before enough amount of cells were obtained.

D.3 Other Cells

Skeletal muscle cells (SkMC, cat SC3500), fetal dermal fibroblasts (5C2300) and human mesenchymal stem cells (MSC, SC7501) from 3H Biomedical were also cultured according to the manufacturer's instruction and evaluated for growth on starch carriers. The MSCs from 3H Biomedical grew a little faster than the ones from Lonza, probably due to a different media but gave similar cell growth score on starch carriers as the Lonza-MSCs The dermal fibroblasts were cultured with serum-free media.

E. Amylase Degradation and Effect on Cells

E.1 Detachment of Cells from Starch Carriers

After 72 hours of cell growth on starch beads culture, degradation and cell release experiments were performed. Two different concentrations of amylase have been tested. Moreover, different additive methods, including Trypsin/EDTA, collagenase, Accumax (were tested in order to improve degradation of carriers and/or detachment of cells into single cells. The commercial product Accumax, which was most effective, combines protease, collagenolytic and DNase activities making it an effective cell aggregate dissociation solution. Moreover, Accumax does not contain mammalian or bacterial-derived products.

E.2 Amylase Activity and Cytotoxicity

Three different amylases have been tested; 1) porcine pancreatic α-amylase Type I-A (A 6255, Sigma), which was used throughout the whole study and in all degradation experiments, 2) human amylase from saliva, which was much less efficient than the porcine pancreatic α-amylase and 3) a bacterially produced amylase (α-Amylase from Bacillus sp, A 6380, Sigma). An α-Amylase from human saliva (A 0521, Sigma) did not appreciably degrade the starch carriers. Two different amylase inhibitors (α-Amylase inhibitor from Triticum aestivum (wheat seed) Type I and Type III, Sigma) could inhibit degradation by serum, but appeared to not be very efficient. When used at 500-1000 U/L only marginal effects were seen (the concentration in the body is 70-300 U/L).

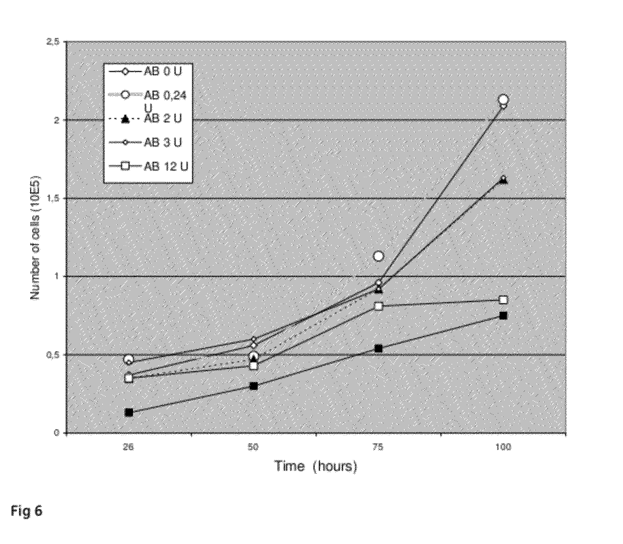

Results suggest that degradation rate is controlled by the number of amylase units/gram carrier and not the concentration of amylase. Thus, a high amylase concentration in low volume gives degradability equal to a low concentration of amylase in a high volume. To assess if amylase at high concentration is toxic to cells a toxicity assay was performed using different concentrations of amylase in media and MSCs cultured in monlayer for four days, changing the media daily. It was found that amylase at concentrations of 12 units/ml or more could inhibit cell growth after 3 days (FIG. 6) but that at levels expected in vivo or in many culture applications amylase did not have major effects on cell viability by standard Trypan Blue assay as cell viabilities were typically above 95% (not shown).

II Experimental Results and Discussion

A. Commercial and Chemically Modified Particles

Commercial Cytodex I, II and Cytodex base matrix (Sephadex G50 type) particles were of normal size and density (GE Healthcare product literature, see above). Test samples of Magle AB starch particles covering a range of amylase degradation susceptibility, by both the Magle AB and GE test methods, (Table 4) had densities of 1.02 to 1.09 g/cm³ measured by density gradient sedimentation in Percoll gradient with density matched to Density Marker Beads.

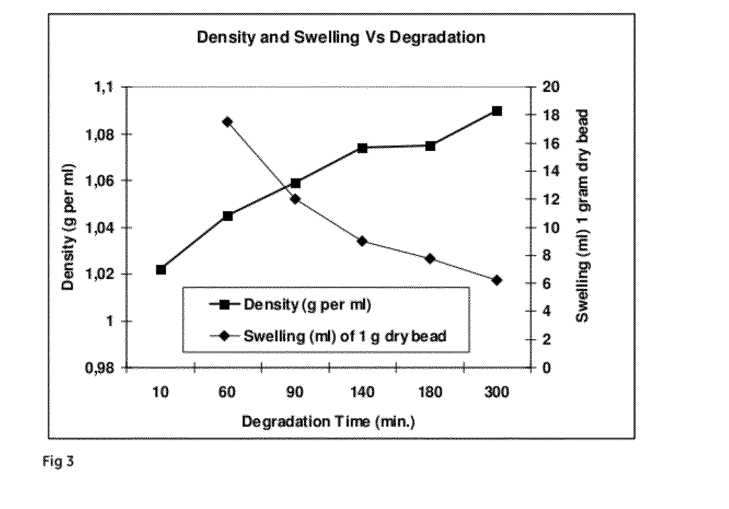

There was good correlation in the results from the two different degradation tests (Table 4) and, based on the hypothesis that density increases with cross linking, there was a direct relation between density and degradation time, and a direct inverse relation between degradation time and swelling which is expected to relate to ability of amylase to diffuse into the hydrogel and enzymatically hydrolyse the gel (FIG. 3)

TABLE 3
Starch Particle Base Matrices

| | | | Density | |
|---|---|---|---|---|
| | Degradation | GE Degradation | (GE QC | |
| Starch bead | Time | Score | method) | Swelling of 1 g |
| batch | (Magle AB) | (0 to 8) | (g/ml) | dry gel in ml. |
| C1A | 140 | 7 | 1.074 | 9.0 |
| C1B | 180 | 7 | 1.075 | 7.8 |
| C2 | 10 | 8 | 1.022 | |
| C3 | 300 | 5 | 1.090 | 6.2 |
| C4A | 90 | 8 | 1.059 | 12.0 |
| C4B | 60 | 8 | 1.045 | 17.5 |

Notes.
1. Density determined using Percoll density gradient and density gradient marker beads.
2. Density of Cytodex 1 commercial cell carrier is 1.076 by same method.

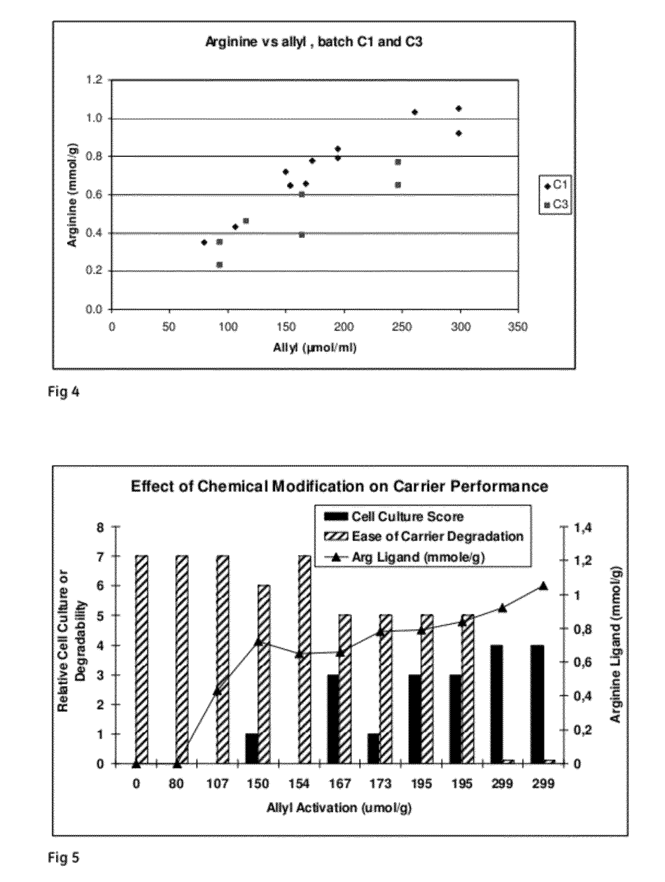

The reaction method shown in FIG. 1 readily allows for control over activation and ligand density. As seen previously for Cytodex base matrices (SE 0802474-7) there was a direct relation between arginine ligand density (coupling efficiency) and allylation with the bifunctional reagent. This is illustrated in FIG. 4 for carrier samples based on starch particle types Cl and C3 (Table 3). Data for allylation and arginine ligand coupling in Cl starch particles of initial density 1.07 are also given in Table 4 where it can be seen that the prototypes studied covered a range of production and performance variables. The densities of the resulting ligand modified starch particles obtained ranged from 1.02 to 1.09 (Table 3) which is similar to the densities of commercial Cytodex carriers (and thus are commensurate with their possible use in large scale stirred bioreactors.

DEAE coupled ligands (Table 5) provided similar grafting densities to AGE coupled ligands as did epichlorohydrin (ECH) coupled arginine ligands (Table 6). Note that activation and ligand analysis methods were as per SE 0802474-7.

B. Cell Culture

B.2 Human Mesenchymal Cells (MSCs)

Results for MSCs are given in Table 4 and FIG. 5 where it can be seen that it is readily possible to develop carriers which offer both good susceptibility to amylase based degradation and cell culture capability. As noted earlier for arginine modified Cytodex (SE 0802474-7) carriers, in regard to both Vero and MSC cell types, there appears to be a minimum surface density of arginine ligands which are required to achieve good cell attachment and growth (FIG. 5). Cell growth also appears to be affected by degree of allylation which is to say unreacted allyl groups which are expected to hydrolyse to hydroxyl groups (SE 0802474-7) (FIG. 5). However there appears to be a significantly broad range of both allylation and ligand coupling where both cell culture and susceptibility to degradation are both significant. FIG. 5 suggests that for various applications it may be possible to tailor culture and degradation susceptibility. The data also suggests that in order to construct an effective culture hydrogel which degrades rapidly it may be better to allylate and ligand couple only the external surface of the hydrogel in the manner of so called “lid” gels used for chromatography. Such an approach may also save on time, reagent cost, and allow for carriers whose degradation rates more closely match those of unmodified starch particles.

TABLE 4
Growth of Human Mesenchymal Stem Cells On, and Amylase Based Degradation For
Allyl Activated, Arginine Coupled Starch Particle Carriers

| | | Allyl | Arginine | Cell culture | Degradation |
|---|---|---|---|---|---|
| Carrier | Prototype Rationale | μmol/ml | mmol/g | Score (0-5) | Score (0-8) |
| 1 | Base Matrix Control | 0 | 0 | 0 | 7 |
| 2. | Low Activation Control | 80 | 0 | 0 | 7 |
| 3 | Low Activation Control 2 | 107 | 0 | 0 | 7 |
| 4 | Low Act'n., Low Ligand 1 | 80 | 0.35 | 0 | 7 |
| 5 | Low Act'n., Low Ligand 2 | 107 | 0.43 | 0 | 7 |
| 6 | Med. Act'n., Low Ligand 3 | 154 | 0.65 | 0 | 7 |
| 7 | Med. Act'n., Med. Ligand 2 | 150 | 0.72 | 1 | 6 |
| 8 | Med. Act'n., Med. Ligand 3 | 167 | 0.66 | 3 | 5 |
| 9 | Med. Act'n., Med. Ligand 4 | 173 | 0.78 | 1 | 5 |
| 10 | High Act'n., Med. Ligand | 195 | 0.79 | 3 | 5 |
| 11 | High Act'n. Med. Ligand 2 | 195 | 0.84 | 3 | 5 |
| 12 | V. High Act'n., High Ligand | 299 | 0.92 | 4 | 0 |
| 13 | V. High Act'n., High Ligand | 299 | 1.05 | 4 | 0 |

Note.
1. Base Matrix C1A was unmodified starch particle with density of 1.07 gram per ml. Rationale was to study four degrees of allyl activation (low, medium, high and very high) and three degrees of ligand coupling (low, medium and high).
2. Relative score for cell culture performance where 0 is no cell culture, 3 is passible performance and 5 is excellent performance.
3. Relative score for amylase based carrier degradation, in vitro, where 0 is no appreciable degradation and 8 is rapid complete degradation. A score of 5 or greater should offer performance suitable for many applications.

TABLE 5
MSC Culture on DEAE and Arginine Modified Starch Carriers.

| Ligand on starch beads | Cells | |
|---|---|---|
| (Ligand density in mmol/g) | attached | Degradation |
| DEAE (3.36) | no | no |
| DEAE (1.29) | no | some beads degrade within ~3 h |
| Arginine (1.03) | yes | no |
| Arginine (0.66) | yes | gradually/day by day/ |
| | | some beads do not degrade at all |

Note.
DEAE ligand concentration on Cytodex 1 is 1.4-1.6 mmol/g.

Cells did not at all attached to starch beads modified with various amounts of DEAE as ligand, using VERO cells or human mesenchymal stem cells as test cells. On the other hand cells did attach and spread on starch beads modified with a broad variety of ligand density when using arginine as ligand (Table 4). In a similar way it was shown that cells did not attach to starch beads when using arginine in combination with epichlorohydrin as coupling reagent (Table 6).

TABLE 6
Results from ECH and Arginine Coupling

| | | | | Relative MSC |
|---|---|---|---|---|
| | Relative | Epoxide | Arginine | Cell Culture |
| Base Matrix | Degration Time | (μmol/ml) | (mmol/g) | Performance |
| C1 | 140 | 178 | 0.52 | Poor |
| C2 | 10 | 15 | 0.52 | Poor |

B.3 Other Cells

The fact that the arginine modified carriers appear suitable for a variety of cell types is interesting given that some cases cells which grow on one carrier surface may not grow on another (e.g. Assessment of stem cell biomaterial combinations for stem cell-based tissue engineering. Neuss, Sabine et al. BioMat, Aachen University, Aachen. Biomaterials 29 (2008) 302 to 313). Apart from MSCs from Lonza we have also cultured MSCs from 3H biomedical (not same media as Lonza's MSCs), skeletal muscle cells and fetal dermal fibroblasts. All cell types that are grown in serum exhibit similar growth scores on starch carriers. However, the fetal fibroblasts, which were cultured under serum free conditions showed better growth on the easily degradable carriers. The reason for this is that serum contains amylase (up to 1 U/ml in 10% serum) that starts to degrade the carriers during culture causing the cells to detach. Thus cells cultured in serum-free conditions grow well on these carriers for a longer period up to 48 hour longer. However, the cells themselves also appear to produce amylase or some other degrading enzyme and when this happens the carriers change in shape, the cells round up and detach.

B.4 Cells on Lid Beads

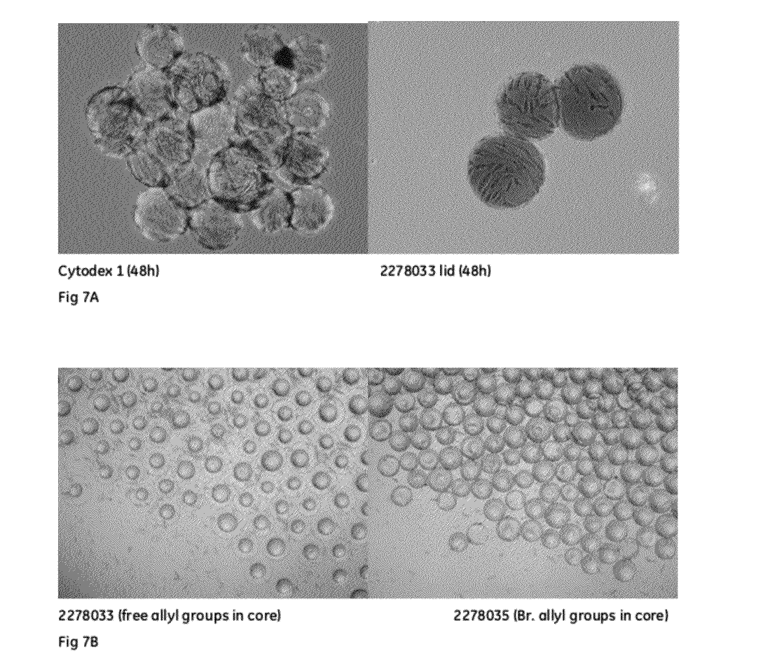

Skeletal and MSCs grew very nicely on lid-protypes (FIG. 7A) shows SkMCs on Cytodex 1 and one of the lid-protypes) with a growth score between 4 and 5 (i.e. better than on Cytodex 1). Two different approaches were made to make the lid-coupling, one with bromated allyl groups in the core, and the other with free allyl groups in core) and these degraded very differently. The first appear to degrade from the inside and the other from the outside (FIG. 7B).

C. Amylase Based Carrier Gel Degradation and Effect on Cells

In some applications one may wish to use carriers that do not readily degrade with amylase but are composed of a novel biocompatible material such as starch. Carrier beads which offer reduced degradation with amylase can still have cell removal effected via trypsinisation. For some C1A based cell carriers which partly degraded by amylase over 4 hours the commercial preparation Accumax allowed for complete degradation of the starch mass single cell release in 5 minutes (incubation according to manufacturers directions). Addition of Accumax at 1 and 2.5 hours, i. e. before appreciable amylase based degradation had little effect on cell recovery which suggests that that Accumax cannot function on its own in this type of application.

1. A method for cell expansion comprising the following steps:
a) adding cells, culture medium and cell culture surface comprising a degradable polysaccharide, having guanidine group containing ligands on its outer surface, to a bioreactor;
b) expanding said cells by adherent cell culture; and
c) aiding the detachment of said cells by exposing them to a polysaccharidase to degrade the culturing surface.

2. The method of claim 1, wherein the culture surface is a microcarrier particle, a slide, a biosensor chip, a disposable tube or bag, or a microtiter plate.

3. The method of claim 1, wherein the degradable polysaccharide is coated to the culture surface.

4. The method of claim 3, wherein coating and the culture surface are made of different material.

5. The method of claim 1, wherein the polysaccharide is dextran or starch and the polysaccharidase is dextranase or amylase.

6. The method of claim 1, wherein the guanidine group-containing ligands are Arginine-ligands, preferably monopeptides or dipeptides comprising at least one arginine residue.

7. The method of claim 1, wherein the ligands are covalently grafted to the culture surface which has been activated with a bifunctional reagent.

8. The method of claim 1, wherein the ligands are attached to the degrading polysaccharide surface via a allylglycidylether or analogous bifunctional reagent which is first coupled to the culture surface, or to the ligand.

9. The method of claim 1, wherein the cultured cells are detached by a method involving polysaccharidase which is not added to the cultured cells environment but occurs spontaneously as a recombinant or normal cell gene product.

10. The method of claim 1, wherein the cell culture surface is a microcarrier comprising starch and the guanidine group containing ligands are Arg-ligands provided in the surface of the microcarrier as a lid.

11. The method of claim 10, wherein the starch is provided as a coating on a microcarrier made of other material than starch.

12. The method of claim 1, wherein the cells are primary cells or stem cells.

13. The method of claim 1, wherein the cells are established cell lines.

14. The method of claim 1, wherein the microcarriers are provided with magnetic particles.


SEQUENCE LISTING (3 sequences)

SEQ ID NO: 1 - protein, 7 aa, Artificial Sequence
  SYNTHETIC OLIGOPEPTIDE
GRGDSPK

SEQ ID NO: 2 - protein, 4 aa, Artificial Sequence
  SYNTHETIC OLIGOPEPTIDE
GGYR

SEQ ID NO: 3 - protein, 5 aa, Artificial Sequence
  SYNTHETIC OLIGOPEPTIDE
RKDVY